(19) United States
(12) Patent Application Publication
(10) Pub. No.: US 2014/0296144 A1
(43) Pub. Date: Oct. 2, 2014

(54) PROCESS FOR THE PREPARATION OF OCTREOTIDE ACETATE

(71) Applicant: Mylan Laboratories, Ltd., Hyderabad (IN)

(72) Inventors: Ananda Kuppanna, Hyderabad (IN); Mallikarjuna Sarma Dokka, Hyderabad (IN); Sreelatha Vanjivaka, Hyderabad (IN); Maria Bhaskar Reddy Komma Reddy, Hyderabad (IN); Bulliraju Kamana, Hyderabad (IN)

(21) Appl. No.: 14/348,063

(22) PCT Filed: Sep. 28, 2012
(86) PCT No.: PCT/IN12/00652

§ 371 Date: Mar. 28, 2014

(30) Foreign Application Priority Data
Sep. 30, 2011 (IN) 3397/CHE/2011

(51) Int. Cl. C07K 1/04; C07K 7/06; C07K 1/14

(52) U.S. Cl. CPC C07K 1/042; C07K 1/14; C07K 7/06
USPC 514/11.1; 530/344

(57) ABSTRACT

The present invention relates to an improved process for large scale production of Octreotide acetate. The invention also relates to pharmaceutical composition of octreotide acetate.

### FIELD OF THE INVENTION

The present invention relates to an improved process for large scale production of octreotide acetate by solid phase synthesis.

### BACKGROUND OF THE INVENTION

Octreotide is an octapeptide that mimics natural somatostatin pharmacologically, though it is a more potent inhibitor of growth hormone, glucagon, and insulin than the natural hormone.

The presence of D-Phe at the N-terminal and an amino alcohol at the C-terminal, along with D-Tryptophan and a cyclic structure makes it very resistant to metabolic degradation.

Octreotide comprises 8 amino acids which has the following structural formula:

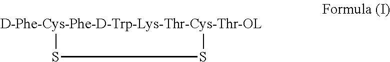

Conventional synthesis of octreotide may be divided into two main approaches, liquid-phase synthesis and solid-phase synthesis.

Octreotide first disclosed in U.S. Pat. No. 4,395,403, in which Octreotide is prepared by solution phase peptide synthesis. The process comprises; removing protected group from peptide; linking together by an amide bond to two peptide unit; converting a function group at the N- or C-terminal; oxidizing a straight chain polypeptide by boron tristrifluoroacetate.

Since all the synthesis steps are carried out in liquid phase, US'403 process is a time-consuming, multi-step synthesis and it is difficult to separate octreotide from the reaction mixtures.

Another solution phase approach described in U.S. Pat. No. 6,987,167 and WO2007110765A2, in which the cyclization of partially deprotected octreotide is carried out in the solution phase using iodine under specific conditions in presence of alcoholic solvents.

U.S. Pat. No. 6,346,601B1, WO2005087794A1 and WO2010089757A2 disclose a process for the preparation of octreotide by hybrid approach i. e synthesis of fragments on solid phase and condensing the obtained fragments in a liquid phase.

U.S. Pat. No. 6,476,186 describes the solid phase synthesis, in which the synthesis of octreotide using Thr(ol)(tBu)-2Cl-trityl resin as starting material, followed by the cleavage of the straight chain peptide from the resin using a strong acid and the formation of the intra-molecular disulfide bond on the completely deprotected octreotide by oxidation using charcoal catalyst.

US20040039161A1 provides a solid phase peptide synthetic method for the preparation of C-terminal alcohols using trichloroacetimidate activated linker, making the required peptide chain on the resin support, cleaving the attached peptide; air oxidation to form said C-terminal amino alcohol containing peptide and a 36.3% yield of octreotide after HPLC purification.

Charcoal oxidation or air oxidation needs longer reaction time and results in low yield. Further, in large scale, the conversion of dithiol to disulfide bond ends in unconverted starting material.

Another solid phase approach describes in Bioconjugate chem. 2009, 20, 1323-1331. This article discloses the process of somatostatin and octreotide analogues using solid phase peptide synthesis with CTC resin.

Journal of Harbin Institute of Technology, 2008, Vol 40 (2), 292-295, discloses the process for the preparation of octreotide using CTC resin. According to this process the obtained octreotide has the purity 70.26% by HPLC.

During the process of peptide bond formation which is mediated by a coupling agent, the carboxylic group of amino acid interacts with the coupling agent to form an activated intermediate, which in turn interacts with the amino group of the next amino acid.

Racemization is a side-reaction that occurs during the preparation of a peptide. In large scale production, the formations of small amounts of epimers are possible. Detection and removal of these impurities are very difficult. This constitutes one of the most serious drawbacks for the implementation of peptides in commercial scale production.

The rate of racemization in peptide synthesis is also the most difficult to measure. The term ‘racemization’ as used herein, refers to partial conversion of one enantiomer of a chiral molecule into its other stereomeric form, or its ‘mirror image’ inversion. As used herein, the term ‘enantiomer’ refers to a stereoisomer of an amino acid that is superimposable with respect to its counterpart only by a complete inversion/reflection of each other.

Carbodiimides are presently the most available and low-cost coupling agents amongst the presently known reagents. The primary reactive species, O-acylisourea, is one of the most reactive species for peptide coupling.

In the last decade, the use of phosphonium and imminium/uronium salts of hydroxybenzotriazole derivatives as peptide coupling agents was introduced. Although these reagents requires at least one equivalent of base is essential for coupling. Racemization was observed with the use of these coupling agents on large scale synthesis especially at the Cys residues.

According to the prior art processes, use of base in building the peptide chain leads to racemization, which results in less purity of resin protected octreotide. The present inventors have developed the process for the preparation of octreotide without base with good yield and purity by, minimizing the racemization at resin protected peptide.

A considerable number of known, naturally occurring small and medium-sized cyclic peptides as well as some of their artificial derivatives and analogs possessing desirable pharmacological properties have been synthesized. However, wider medical use is often hampered due to complexity of their synthesis and purification. Therefore, improved methods for making these compounds in simple, lesser steps, and at lesser cost are desirable. The present process for the preparation of octreotide is feasible in large scale production with high yield and purity. According to the present invention is that the attachment of Fmoc-Thr-OL to 2-CTC resin is better in terms of loading, yield and purity when compared to Fmoc-Thr(tBu)-OL.

### SUMMARY OF THE INVENTION

The present invention relates to an improved process for large scale production of octreotide acetate.

The main aspect of the present invention relates to an improved process for obtaining octreotide acetate by means of solid phase synthesis using 2-chloritrityl chloride resin. Coupling of the appropriate protected amino acid in a required sequence, cleavage and deprotection, oxidation of the dithiol to disulfide, followed by purification (Scheme-1 & 2).

Another aspect of the present invention is to provide a process for the preparation of octreotide acetate by means of hybrid approach i.e. the preparation of hexapeptide on solid phase and coupling with the dipeptide in the solution phase, oxidation of the dithiol followed by cleavage and deprotection (Scheme-3).

Yet another aspect of the present invention is to provide a process for the preparation of octreotide acetate using modified Wang resin as a solid support (Scheme-4).

Yet another aspect of the present invention is to provide a process for the preparation of octreotide acetate by making appropriate fragments and condensing them in solution phase (Scheme-5).

Yet another aspect of the present invention is to provide a pharmaceutical composition of octreotide acetate.

The schematic description of the process is as shown in scheme-1

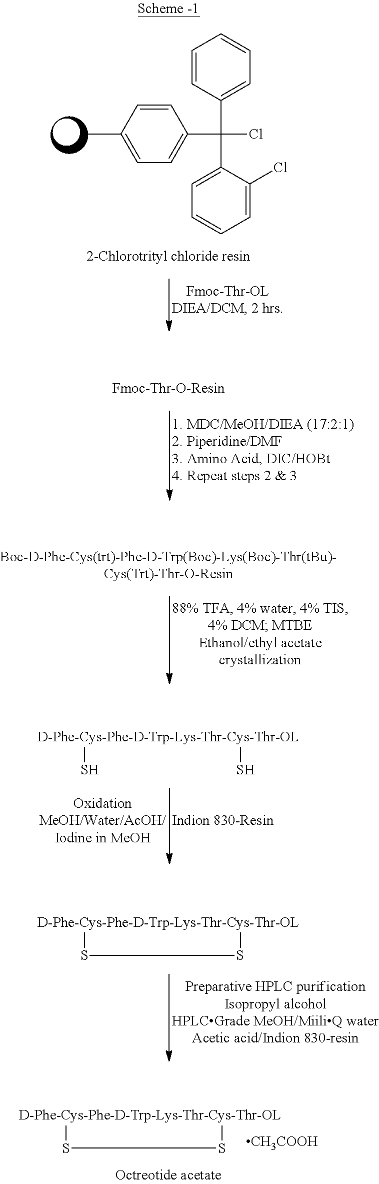

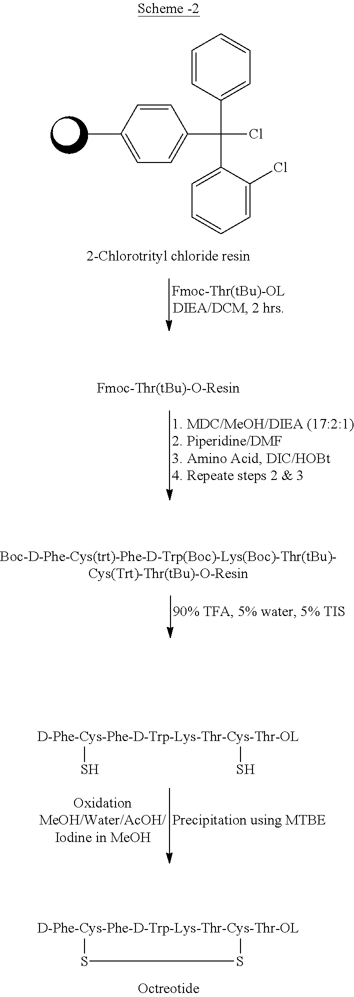

The alternative processes for the synthesis of octreotide have shown schematically in scheme-3 to scheme-5.

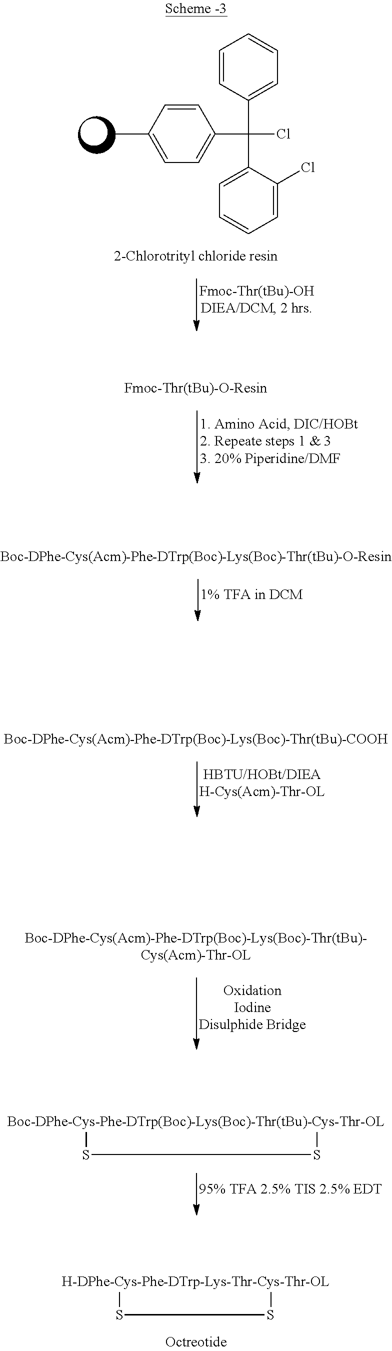

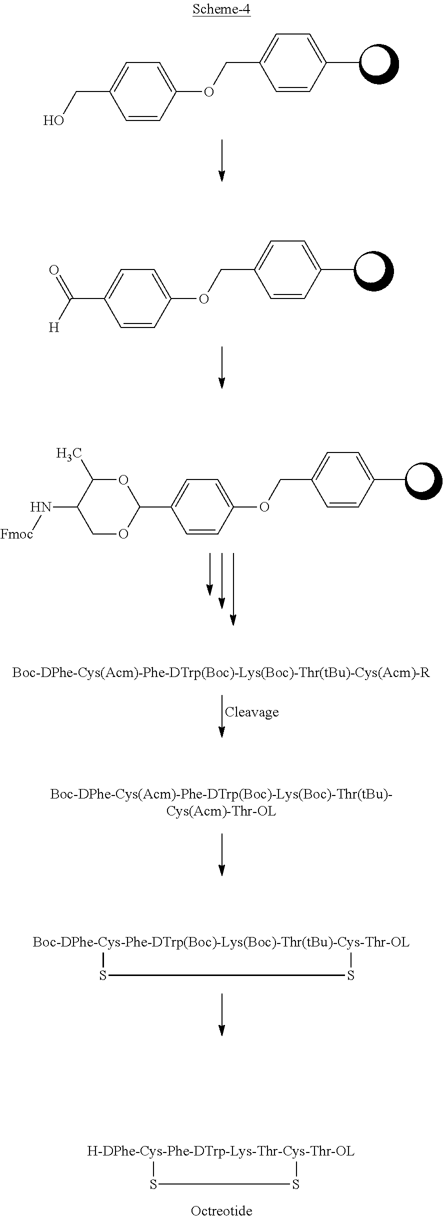

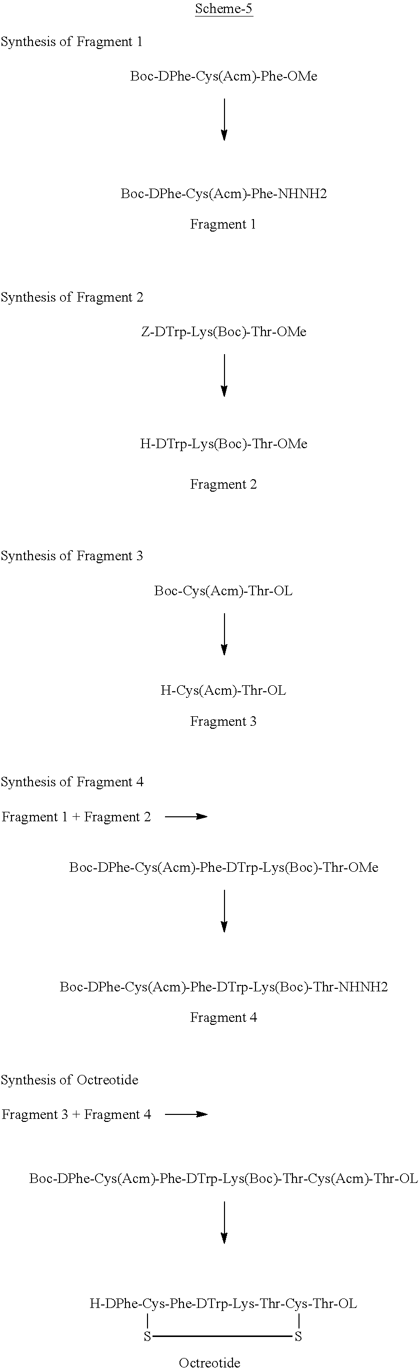

### DETAILED DESCRIPTION OF THE INVENTION

The present invention relates to an improved process for large scale production of octreotide acetate. The invention also relates to pharmaceutical composition of octreotide acetate.

Abbreviations:
AcOH acetic acid
t-Bu tert-butyl
DCC N,N′-dicyclohexyl carbodiimide
DCM dichloromethane
DIC N,N′-diisopropylcarbodiimide
DMF N,N′-Dimethylformamide
DIEA Diisopropylethylamine
2-CTC resin 2-Chloritrityl chloride resin
Fmoc 9-fluorenylmethoxycarbonyl
HOBt N-hydroxybenzotriazole
HBTU O-Benzotriazole-N,N,N′,N′-tetramethyl-uronium-hexafluoro-phosphate
MTBE Methyl tert-butyl ether
SPPS solid phase, peptide synthesis
Phe Phenylalanine
Cys Cystine
Trp Tryptophan
Lys Lysine
Thr Threonine
TFA Trifluoroacetic acid
TIS Triisopropylsilane

One embodiment of the present invention is to provide a process for the preparation of octreotide acetate comprising the steps of:
a. anchoring eighth protected N-terminal amino alcohol to a resin,
b. capping the resin obtained in step a),
c. selectively deprotecting the amino group,
d. coupling the carboxyl terminus of the next N-protected amino acid to the amine group in presence of a coupling reagent,
e. repeating steps c) and d) to synthesize the desired peptide sequence,
f. cleaving the peptide with a cocktail mixture from the resin to isolate linear chain dithiol peptide,
g. oxidizing the linear chain dithiol peptide into disulphide bridge peptide,
h. quenching the excess of iodine, followed by salt exchange simultaneously using anion exchange resin, and
i. purifying the obtained compound to isolate octreotide acetate.

According to the present invention, the resin used is 2-chlorotrityl chloride resin. The resin undergoes swelling in the presence of a solvent selected from dichloromethane, N,N-dimethylformamide, N-methyl-2-pyrrolidone or mixtures. The swelled resin is treated with N-terminus protected amino alcohol in the presence of DIEA for the etherification of the 2-CTC resin.

According to the present invention, the eight amino alcohol anchored to the resin is typically Fmoc-L-Thr-OL, wherein the amino terminus is blocked by a protecting group preferably, Fmoc-group. The amount of protected amino alcohol used in the anchoring is normally in excess equivalents, preferably 1.0 to 3.0 equivalents with respect to the resin loading capacity, more preferably 1.5 to 3.0 equivalents of Fmoc-Thr-OL and the quantity of DIEA used in this step varies from 3.0 to 6.0 equivalents with respect to protected amino alcohol, preferably 3.0 equivalents:

The attachment of Fmoc-Thr-OL to the resin is confirmed by UV for determining resin substitution in SPPS, which involves the treatment of known quantity of Fmoc-loaded resin with piperidine in DMF and the released amount of dibenzofulvene-piperidine adduct is measured spectrophotometrically.

After anchoring the first protected amino alcohol, before proceeding to the next steps, the unreacted linkers on the resin are appropriately protected (capped) in order to avoid the undesired peptide chain formation. The reagent used for the capping such as methanol/DIEA/DCM in the ratio (2:1:17).

The deprotection of the amino acid attached to the resin is done selectively in the presence of a nucleophilic base such as 20% piperidine in presence of a solvent. The solvent is selected from N,N-dimethylformamide, methylene chloride, tetrahydrofuran, N-methyl pyrrolidine or mixture thereof.

The process of selective deprotection of protected amino acid attached to the resin or in the peptide chain is carried out at a temperature in the range of 5-30° C. Preferred temperature for the selective deprotection is 20-25° C., however it may vary from one amino acid to another amino acid. The process of selective deprotection further comprises washing the deprotected amino acid with a suitable solvent such as dichloromethane or dimethylformamide or mixture thereof to remove residual reagents and byproducts.

The coupling efficiency after each coupling step is monitored during the synthesis by means of a Kaiser test or any other suitable test. If the coupling efficiency is low, the steps of individual coupling are to be repeated prior to the deprotection and coupling with next amino acid sequence.

According to the present invention, deprotected the amino group is coupled with next N-protected amino acid in a solvent in presence of a coupling reagent.

The solvent used for the coupling reaction is selected from dichloromethane, tetrahydrofuran, dimethylformamide, N-methylpyrolidone or mixture thereof. The coupling agent used for the coupling of the amino acids is selected from DIC/6-Cl-HOBt, DIC/HOBt, HBTU/HOBt/DIEA or DIC/Oxyma.

According to the present invention, racemization of Cysteine is minimized by coupling with DIC/HOBt in presence of NMP. Whereas the coupling in the presence of DMF solvent sometimes gave racemization in higher levels, this may be due to contamination of DMF with traces of amines. The coupling of the reaction using HBTU/HOBt/DIEA on large scale production gave the racemization levels around 4.0%.

According to the present invention after the completion of the reaction, the resin is optionally washed with solvents such as DMF and DCM to remove residual reagents and byproducts. The process is repeated if desired and before proceeding to next step.

According to the present invention, the cleavage and global deprotection (a process for deprotecting the protected amino acid in the peptide, which has additional functional groups) of the peptide is carried out with a cocktail mixture. The cleavage of the peptide from the resin involves treating the protected peptide anchored to the resin with an acid having at least one scavenger. The acid utilized in the cleavage reagent is TFA. The amount of TFA used for the purpose of cleavage of peptide from the resin and global deprotection in the cocktail mixture may range from 80-90%. The scavengers used are selected from TIS, water or in any combination thereof. The particular Cocktail mixture used for the cleavage of the peptide from resin is 88% TFA, 4% water, 4% TIS and 4% DCM. The temperature for the cleavage and global deprotection is carried out at about 10-30° C., preferably at 25° C. After completion of the reaction, the mixture is added to pre-cooled ether solvent at about −5 to 5° C. The obtained crude octreotide is filtered and washed with acetic acid to get acetate salt.

According to the present invention the use of excess of inexpensive anion exchange resin to the oxidized peptide from the aqueous acetic acid/methanol solution affords quantitative removal of iodine and other color impurities. This improves, the resin life time of expensive chromatography media that is used in preparative HPLC column during the purification of peptide. The peptide is free from iodine and ready for direct purification. Further it is very useful for the conversion of TFA salt to acetate in situ.

According to present invention, the peptide thiol is subjected to oxidation into disulphide bridge in methanol (small amount of acetic acid and water) containing iodine. The disulphide bond also obtained in air oxidation or K₃Fe(CN)₆ under basic conditions. The peptide formed after oxidation is subjected to passing through anion exchange resin to quench the excess iodine and for the formation of octreotide acetate. The isolation of octreotide acetate is carried by evaporating methanol and precipitating with ether solvent to get octreotide as a solid. Ether solvents that are used include but are not limited to diethyl ether, methyl tert-butyl ether, isopropyl ether or combinations thereof.

According to the present invention, the resultant octreotide acetate purification process is carried out on preparative HPLC using Kromasil C-18, 10 micron (50×250 mm) and eluting with a solvent system of 0.2% acetic acid in water (A) and 0.2% acetic acid in methanol (B). A linear gradient of 20-60% B is used at a flow rate of 80 ml/min and detection at 220 nm.

Yet another embodiment of the present invention is to provide a process for the preparation of octreotide acetate by means of hybrid approach i.e. the preparation of hexapeptide on solid phase and coupling with the dipeptide in the solution phase.

Yet another embodiment of the present invention is to provide a process for the preparation of the octreotide acetate using modified Wang resin as a solid support.

Yet another embodiment of the present invention is to provide a process for the preparation of the octreotide acetate by making appropriate fragments and condensing them in solution phase.

Yet another embodiment of the present invention is to provide a pharmaceutical composition comprising octreotide acetate and pharmaceutically acceptable carrier.

The following example is provided to illustrate the process of the present invention. How ever, they are not intended to limit the scope of an invention.

### EXAMPLES

Stage-I: Preparation of Protected Octreotide Anchored to 2-CTC Resin

Method-1:

Octreotide was synthesized manually on 2-chlorotrityl chloride resin (substitution 0.90 mmol/g) by standard Fmoc solid phase synthesis strategy. The resin was soaked in the mixture of MDC and DMF for the swelling. Fmoc-Thr(tBu)-OL was treated with the swelled 2-CTC resin in DCM in the presence of DIEA and substitution level was determined by weight gain measurements and also by UV Method. After the coupling of the first amino acid onto the resin, the un-reacted linkers on the resin (polymer) are protected, to avoid the undesired peptide chain formation, with a solution of 5% DIEA and 10% methanol in DCM. This process of capping is performed after anchoring the first protected amino acid to the resin. The complete synthesis was achieved by stepwise coupling of Fmoc-Amino acids to the growing peptide chain on the resin. All the couplings were carried out in DMF. The N-terminal Fmoc group was removed with 20% (V/V) piperidine in DMF. The couplings were performed by dissolving the Fmoc-Amino acid (2 eq.) and HOBt (2 eq.) in DMF. The solution was cooled on ice and then DIC (2 eq.) was added. The reaction mixture was added to the resin and allowed to react for 2 hrs. The efficiency of the coupling was monitored using the Kaiser Ninhydrin test. The coupling step was repeated if Kaiser test was found positive. The sequence of addition for the synthesis of Octeriotide was Fmoc-Cys(Trt), Fmo-Thr(tBu), Fmoc-Lys(Boc), Fmoc-Trp(Boc), Fmoc-Phe, Fmoc-Cys(Trt), Boc-D-Phe.

Method-2:

Octreotide was synthesized manually on 2-chlorotrityl chloride resin (substitution 0.90 mmol/g) by standard Fmoc solid phase synthesis strategy. The resin was soaked in the mixture of MDC and DMF for the swelling. Fmoc-Thr-OL was treated with the swelled 2-CTC resin in DCM in the presence of DIEA and substitution level was determined by weight gain measurements and also by UV Method. After the coupling of the first amino acid onto the resin, the un-reacted linkers on the resin (polymer) are protected, to avoid the undesired peptide chain formation, with a solution of 5% DIEA and 10% methanol in DCM. This process of capping is performed after anchoring the first protected amino acid to the resin. The complete synthesis was achieved by stepwise coupling of Fmoc-Amino acids to the growing peptide chain on the resin. All the couplings were carried out in DMF. The N-terminal Fmoc group was removed with 20% (VN) piperidine in DMF. The couplings were performed by dissolving the Fmoc-Amino acid (2 eq.) and HOBt (2 eq.) in DMF. The solution was cooled on ice and then DIC (2 eq.) was added. The reaction mixture was added to the resin and allowed to react for 2 hrs. The efficiency of the coupling was monitored using the Kaiser Ninhydrin test. The coupling step was repeated if Kaiser test was found positive. The sequence of addition for the synthesis of Octeriotide was Fmoc-Cys(Trt), Fmo-Thr(tBu), Fmoc-Lys(Boc), Fmoc-Trp(Boc), Fmoc-Phe, Fmoc-Cys(Trt), Boc-D-Phe.

Stage-II: Cleavage of Peptide from Resin Along with Global Deprotection

The peptide resin (200 g, obtained in stage I) was swelled in DCM (500 mL) for 15 to 20 minutes under nitrogen at 25-30° C. The cocktail mixture (2.0 L-TFA (1.8 L), water (80 mL) DCM (80 mL) and TIPS (80 mL)) was charged to the resin at 25-30° C. and the obtained reaction mixture was stirred for 2.5 hours at 25-30° C. under nitrogen atmosphere. The reaction mixture was filtered and washed the resin with TFA (250 mL). The obtained filtrate was charged into cold MTBE (4 L, pre-cooled to a temperature of 0-5° C.) under stirring and allowing the temperature to rise more than 5° C. The reaction mixture was stirred for 45-75 minutes at 0-5° C. The obtained suspension was filtered, washed the solid with MTBE (5 L) and dried the solid under nitrogen. The product was stir with 5%ethanol in ethyl acetate at 25-30° C. Filtered the product, wash with ethyl acetate and dried under vacuum to obtain a desired product

Stage-III: Disulphide Bridge Formation

The free thiol (100 g) obtained above is dissolved in methanol (22.0 L) with small amount of acetic acid and water (4.5 L) and stirred. Iodine solution (20 gm iodine in 500 mL methanol) was added to the reaction mass slowly up to yellow color persists. The reaction was maintained for another 2 hrs, and the excess iodine quenched with Indion 830-S Resin (900 g) and filtered the resin. The filtrate was evaporated and precipitated using MTBE or directly taken the solution for purification using preparative HPLC.

Stage-IV: Preparative HPLC Purification

Method-1:

The crude disulphide bridge peptide was purified on a preparative reverse phase HPLC system using Kromasil C-18, 10 micron (50×250 mm), and eluting with a solvent system of 0.2% acetic acid in water (A) and 0.2% acetic acid in methanol (B). A linear gradient of 20-60% B was used at a flow rate of 80 mUmin and detection at 220 nm.

The octreotide was eluted at around 25% methanol. The fractions were collected at regular intervals and assayed by HPLC to determine the purity of fractions. The desired purities fractions were pooled together and evaporated using Rota evaporator. The aqueous layer was lyophilized to isolate octreotide acetate

Method-2:

The crude disulphide bridge peptide was purified on a preparative reverse phase HPLC system using Kromasil C-18, 10 micron (50×250 mm) and eluting with a solvent system of 0.4% acetic acid in water (A) and methanol (B). A linear gradient of 25-60% B was used at a flow rate of 80 mUmin and detection at 220 nm.

The octreotide was eluted at around 25% methanol. The fractions are collected at regular intervals and are assayed by HPLC to determine the purity and fractions. The desired purities may be pooled together and were evaporated using Rota evaporator. The aqueous layer was lyophilized to isolate octreotide acetate

1-9. (canceled)

10. A process for preparing octeotide of formula (I) 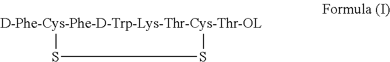
or a pharmaceutically acceptable salt thereof, comprising the steps of:
a. anchoring eighth protected terminal amino alcohol to a resin,
b. capping the resin obtained in step a),
c. selectively deprotecting the amino group,
d. coupling the carboxyl terminus of the next N-protected amino acid to the amino group in the presence of a coupling reagent,
e. repeating steps c) and d) to form a peptide sequence,
f. cleaving the peptide with a cocktail mixture from the resin to isolate a linear chain dithiol peptide,
g. oxidizing the linear chain dithiol peptide into a disulphide bridge peptide using iodine,
h. quenching any excess of iodine, followed by a simultaneous salt exchange using an anion exchange resin, and
i. purifying the obtained compound to isolate the octreotide or the pharmaceutically acceptable salt thereof.

11. The process according to claim 10, wherein the pharmaceutically acceptable salt of octreotide is the acetate.

12. The process according to claim 10, wherein the resin of step a) is a 2-chlorotrityl chloride resin.

13. The process according to claim 10, wherein the deprotection of step c) is carried out in the presence of a nucleophilic base and a solvent.

14. The process according to claim 13, wherein the nucleophilic base is 20% piperidine.

15. The process according to claim 13, wherein the solvent is selected from the group consisting of dichloromethane, methyl tert-butyl ether, tetrahydrofuran, N,N-dimethylformamide, N,N-dimethylacetamide, N-methyl-2-pyrrolidone and mixtures thereof.

16. The process according to claim 10, wherein the coupling agent used for the coupling step of step d) is selected from the group consisting of diisopropylcarbodiimide (DIC)/6-chloro-l-hydroxybenzotriazole (6-Cl-HOBt), DIC/1-hydroxybenzotriazole (HOBt), O-(benzotriazol-1-yl)-N,N,N′,N′-tetramethyluronium hexafluorophosphate (HBTU)/HOBt/N,N-Diisopropylethylamine (DIEA) and DIC/ethyl 2-cyano-2-(hydroxyimino)acetate (Oxyma).

17. The process according to claim 10, wherein the cocktail mixture according to step 0 consists of trifluoroacetic acid (TFA), triisopropylsilane, dichloromethylene (DCM) and water.

18. The process according to claim 10, wherein the oxidation in step g) is carried out in the presence of methanol, acetic acid and water containing iodine.

19. A pharmaceutical composition comprising octreotide acetate obtained by the process of claim 10 and at least one pharmaceutically acceptable carrier.
